(19) United States
(12) Patent Application Publication
(10) Pub. No.: US 2016/0106889 A1
(43) Pub. Date: Apr. 21, 2016

(54) NOVEL CHAMBER FOR THE COLLECTION OF LIPOASPIRATE

(71) Applicants: Bradford A. Conlan, Solana Beach, CA (US); Priya Sridhar, San Diego, CA (US); Thomas Fuller, Dubuque, IA (US)

(72) Inventors: Bradford A. Conlan, Solana Beach, CA (US); Priya Sridhar, San Diego, CA (US); Thomas Fuller, Dubuque, IA (US)

(21) Appl. No.: 14/745,279

(22) Filed: Jun. 19, 2015

Related U.S. Application Data
(60) Provisional application No. 62/015,259, filed on Jun. 20, 2014.

(51) Int. Cl. A61M 1/00

(52) U.S. Cl. CPC A61M 1/0001; A61M 2202/09; A61M 2205/7545; A61M 2207/00

(57) ABSTRACT

The present invention discloses a tissue collection device and the methods of making and using the same. The tissue collection device comprises a vacuum chamber or vacuum lid in conjunction with a disposable tissue filtration system. The vacuum chamber or vacuum lid has a mechanism to allow the vacuum chamber or vacuum lid to receive and open to the disposable tissue filtration system and re-seal to allow tissue/fluid to be aspirated directly in the disposable tissue filtration system via an external vacuum source.

### CROSS-REFERENCE TO RELATED APPLICATION

The present application claims the benefit of U.S. provisional application No. 62/015,259, filed on Jun. 20, 2014, the teaching of which is incorporated herein by reference in its entirety.

### FIELD OF THE INVENTION

Embodiments disclosed herein generally relate to tissue collection chambers and methods of making and using the same.

### BACKGROUND OF THE INVENTION

The transfer of adipose tissue to various regions of the body is a relatively common cosmetic, therapeutic and structural procedure involving the harvest of adipose tissue from one location and re-implantation of the harvested and, oftentimes processed tissue, in another location (see Coleman 1995; and Coleman 2001). While being largely used for repair of small cosmetic defects such as facial folds, wrinkles, pock marks and divots; the transfer of adipose tissue has recently been used for cosmetic and/or therapeutic breast augmentation and reconstruction (Bircoll and Novack 1987; and Dixon 1988), and augmentation of the buttocks (Cardenas-Camarena, Lacouture et al. 1999; de Pedroza 2000; and Peren, Gomez et al. 2000).

In the past, adipose tissue grafts and methods of adipose tissue transfer have been plagued with difficulties and side effects including necrosis, absorption of the implant by the body, infection (Castello, Barros et al. 1999; Valdatta, Thione et al. 2001), calcifications and scarring (Huch, Kunzi et al. 1998), inconsistent engraftment, (Eremia and Newman 2000), lack of durability, and other problems arising from lack of neovascularization and necrosis of the transplanted tissue. One of the biggest challenges in adipose tissue transfer is absorption of the implant by the body and volume retention of adipose tissue grafts following transfer. When adipose tissue is harvested or washed, the space between individual pieces of harvested adipose tissue is filled by liquid (e.g., water, blood, tumescent solution, oil). When this tissue/fluid mixture is implanted into a recipient the liquid portion is rapidly absorbed by the body resulting in loss of volume. The process by which the amount of fluid is removed from the tissue/fluid mixture is frequently referred to as “drying the adipose tissue” or “dehydrating the adipose tissue”. The content of red and white blood cells and the like within an adipose tissue graft can also significantly affect the volume of graft retained after graft transplantation, due to induction or exacerbation of an inflammatory response. Another aspect of tissue retention relates t the amount of lipid within the adipose tissue graft. It understood that the presence of free lipid (meaning lipids released from dead or damaged adipocytes; also referred to as oil) in adipose tissue grafts can result in induction or exacerbation of an inflammatory response with substantial phagocytic activity and consequent loss of graft volume.

It is also known that mixing unprocessed adipose tissue with a concentrated population of adipose-derived regenerative cells overcomes many of the problems associated with adipose tissue grafts and adipose tissue transfer, as described above. Specifically, supplementing unprocessed adipose tissue with concentrated populations of adipose-derived cells comprising adipose-derived stem cells increases the weight, vascularization, and retention of fat grafts. (See U.S. Pat. No. 7,390,484 and co-pending U.S. Patent Application Publication No. 2005/0025755, herein expressly incorporated by reference in their entireties). Adipose tissue fragments supplemented, or mixed, with a concentrated population of cells including adipose-derived stem cells exhibit improved neoangiogeneis and perfusion in grafts when compared to unsupplemented grafts of adipose tissue alone in animal models. Further, adipose tissue grafts supplemented with adipose-derived regenerative cells that comprise adipose derived stem cells show increased graft retention and weight over time, when compared to unsupplemented grafts. (See U.S. Patent Application Publication No. 2005/0025755). Further, the processing of adipose tissue in a closed, sterile fluid pathway greatly reduces the chance of infection. The improvement in autologous transfer of adipose tissue seen in the animal models described above has also been replicated in human clinical studies. Nevertheless, the isolation and purification of concentrated populations of adipose-derived regenerative cells comprising adipose-derived stem cells (ADSCs), usually involves a series of washing, digestion, filtration and/or centrifugation steps, which can reduce the yield of viable cells, require mechanical equipment and specialized clinicians, and/or can compromise the quality, appearance, longevity, hydration or efficacy of the graft.

To address the above mentioned issues and problems, U.S. patent application publication Nos. 2010/0279405 and 2013/0164731 describe tissue collection devices with limited successes. Therefore, there is a need for additional approaches to prepare and optimize adipose tissue grafts and implants and to isolate and/or concentrate adipose-derived regenerative cells.

The embodiments described below address the above identified issues and needs.

### SUMMARY OF THE INVENTION

In one aspect of the present invention, it is provided a tissue collection device. The device comprises a vacuum chamber or vacuum lid in conjunction with a disposable tissue filtration system. The vacuum chamber or vacuum lid has a mechanism to allow the vacuum chamber or vacuum lid to receive and open to the disposable tissue filtration system and re-seal to allow tissue/fluid to be aspirated directly in the disposable tissue filtration system via an external vacuum source.

In some embodiments of the invention tissue collection device, optionally in combination with any or all the various embodiments described herein, the vacuum chamber is a disposable vacuum chamber.

In some embodiments of the invention tissue collection device, optionally in combination with any or all the various embodiments described herein, the disposable tissue filtration system is physically bonded into the disposable vacuum chamber such that the tissue collection device as a whole is a single use, disposable device.

In some embodiments of the invention tissue collection device, optionally in combination with any or all the various embodiments described herein, the vacuum chamber or vacuum lid is formed of a metal material.

In some embodiments of the invention tissue collection device, optionally in combination with any or all the various embodiments described herein, the vacuum chamber or vacuum lid is formed of stainless steel.

In some embodiments of the invention tissue collection device, optionally in combination with any or all the various embodiments described herein, the vacuum chamber is formed of a sterilizable and disposable material.

In some embodiments of the invention tissue collection device, optionally in combination with any or all the various embodiments described herein, the disposable material is a polymer.

In some embodiments of the invention tissue collection device, optionally in combination with any or all the various embodiments described herein, the polymer is poly(vinyl chloride) (PVC) or polycarbonate.

In some embodiments of the invention tissue collection device, optionally in combination with any or all the various embodiments described herein, the vacuum chamber is formed of a sterilizable and disposable material.

In some embodiments of the invention tissue collection device, optionally in combination with any or all the various embodiments described herein, the disposable material is a polymer.

In some embodiments of the invention tissue collection device, optionally in combination with any or all the various embodiments described herein, the polymer is poly(vinyl chloride) (PVC) or polycarbonate.

In some embodiments of the invention tissue collection device, optionally in combination with any or all the various embodiments described herein, the disposable tissue filtration system is a disposable fat tissue filtration system.

In some embodiments of the invention tissue collection device, optionally in combination with any or all the various embodiments described herein, the disposable tissue filtration system is a disposable fat tissue filtration system.

In some embodiments of the invention tissue collection device, optionally in combination with any or all the various embodiments described herein, the disposable tissue filtration system is a disposable fat tissue filtration system.

In a second aspect of the present invention, it is provided a method of forming a tissue collection device. The method comprises:

providing a vacuum chamber or vacuum lid,
providing a disposable tissue filtration system, and
forming the tissue collection device,

wherein the vacuum chamber or vacuum lid has a mechanism to allow the vacuum chamber or vacuum lid to receive and open to the disposable tissue filtration system and re-seal to allow tissue/fluid to be aspirated directly in the disposable tissue filtration system via an external vacuum source.

In some embodiments of the invention method, optionally in combination with any or all the various embodiments described herein, the tissue collection device is according to any of the various embodiments disclosed herein.

In a further aspect of the prevention invention, it is provided a method, which method comprising:

providing a tissue collection device, and
collection tissue via the tissue collection device,

wherein the tissue collection device is according to any of the various embodiments disclosed herein.

### BRIEF DESCRIPTION OF THE DRAWINGS

FIG. 1 shows an embodiment of a tissue collection device of invention.

FIGS. 2a-2c show embodiments of a tissue collection device of invention at three stages of a tissue collection operation: a) the tissue collection device prior to tissue collection, b) the tissue collection device in about half way of a tissue collection operation, and c) the tissue collection device at the end of a tissue collection operation.

FIGS. 3a and 3b show embodiments of the vacuum connection mechanisms of the tissue collection device of invention.

FIG. 4a shows an invention Puregraft 850 ml bag filled with adipose tissue. FIG. 4b shows a Puregraft 850 ml in full set up on a canister with a vacuum lid, the canister bearing volume marks.

FIG. 5 shows the connection ports of a Puregraft 850 ml bag, which include an auxiliary (Aux) port, drain port, an inlet port, and a tissue port.

FIG. 6 shows the top side of a vacuum lid, which includes port to allow insertion of adaptors to vacuum tubing, patient tubing, and waste tubing.

FIG. 7 shows the underside of a vacuum lid, which shows Aux port, drain port, an and a tissue port, and operation notes.

FIG. 8 shows instructions to attach the drain port of a Puregraft 850 ml bag to a vacuum lid.

FIG. 9 shows the step to attach the tissue port of a Puregraft 850 ml bag to a vacuum lid.

FIG. 10 shows that a Puregraft 850 ml bag is inserted into a 3000 cc canister with Lid attached.

FIG. 11 shows an image of a Puregraft 850 ml bag in a canister.

FIG. 12 again shows another image of a Puregraft 850 ml bag in a canister.

FIG. 13 shows a Puregraft 850 ml bag in a canister on a drapped May Stand.

FIG. 14 shows a Puregraft 850 ml bag in a canister on a drapped May Stand at the foot of a patient's bed between the Liposuction machine and the surgeon's cannula.

FIG. 15a shows a Puregraft 850 ml bag filled with adipose tissue indication to attach drain port to drain port. FIG. 15b shows an indication to attach drain aux cap to port.

FIG. 16a shows a Puregraft 850 ml bag filled with approximately 800 ml pre-washed adipose tissue. FIG. 16b shows a Puregraft 850 ml bag filled with approximately 500 ml washed adipose tissue.

### DETAILED DESCRIPTION OF THE INVENTION

In one aspect of the present invention, it is provided a tissue collection device. The device comprises a vacuum chamber in conjunction with a disposable tissue filtration system. The vacuum chamber has a mechanism to allow the vacuum chamber to receive and open to the disposable tissue filtration system and re-seal to allow tissue/fluid to be aspirated directly in the disposable tissue filtration system via an external vacuum source.

In some embodiments of the invention tissue collection device, optionally in combination with any or all the various embodiments described herein, the vacuum chamber is a disposable vacuum chamber.

In some embodiments of the invention tissue collection device, optionally in combination with any or all the various embodiments described herein, the disposable tissue filtration system is physically bonded into the disposable vacuum chamber such that the tissue collection device as a whole is a single use, disposable device.

In some embodiments of the invention tissue collection device, optionally in combination with any or all the various embodiments described herein, the vacuum chamber is formed of a metal material.

In some embodiments of the invention tissue collection device, optionally in combination with any or all the various embodiments described herein, the vacuum chamber is formed of stainless steel.

In some embodiments of the invention tissue collection device, optionally in combination with any or all the various embodiments described herein, the vacuum chamber is formed of a sterilizable and disposable material.

In some embodiments of the invention tissue collection device, optionally in combination with any or all the various embodiments described herein, the disposable material is a polymer.

In some embodiments of the invention tissue collection device, optionally in combination with any or all the various embodiments described herein, the polymer is poly(vinyl chloride) (PVC) or polycarbonate.

In some embodiments of the invention tissue collection device, optionally in combination with any or all the various embodiments described herein, the vacuum chamber is formed of a sterilizable and disposable material.

In some embodiments of the invention tissue collection device, optionally in combination with any or all the various embodiments described herein, the disposable material is a polymer.

In some embodiments of the invention tissue collection device, optionally in combination with any or all the various embodiments described herein, the polymer is poly(vinyl chloride) (PVC) or polycarbonate.

In some embodiments of the invention tissue collection device, optionally in combination with any or all the various embodiments described herein, the disposable tissue filtration system is a disposable fat tissue filtration system.

In some embodiments of the invention tissue collection device, optionally in combination with any or all the various embodiments described herein, the disposable tissue filtration system is a disposable fat tissue filtration system.

In some embodiments of the invention tissue collection device, optionally in combination with any or all the various embodiments described herein, the disposable tissue filtration system is a disposable fat tissue filtration system.

In some embodiments, the vacuum chamber is able to house a flexible filtration system and allows the user to collect aspirated tissue/fluid in a closed manner. The device allows the user to connect a vacuum source/pump source/any aspiration source to the exterior of the device and allows aspirated tissue to be moved directly into the internally housed filtration system. The device allows fluid and tissue to be inserted into the flexible filtration system and acts as a sealed chamber that prevents the flexible filtration system from compression, overexpansion and bursting.

In some embodiments, the device also has a secondary port that allows aspirated contaminants to exit the filtration system.

In some embodiments, the vacuum chamber is made of components and materials (Stainless Steel, Autoclaveable plastics) that allow the system to be cleaned and sterilized multiple times and thus be used in multiple surgical procedures. This version the device is used in conjunction with a disposable fat filtration system (Puregraft System) each time the device is used. The novel vacuum chamber can be opened, disposable Puregraft System inserted and then re-sealed to allow tissue/fluid to be aspirated directly in the Puregraft system via an external vacuum/pump/source.

In some embodiments, the vacuum chamber is a single use, sterile, device that is made of materials that can be sterilized (PVC, polycarbonate). The device is disposed of after each use.

In some embodiments, the tissue collection device is an all-in -one-device that has the fat filtration system physically bonded into a disposable vacuum chamber, packaged sterile and is used a single time.

The invention device differs from prior art devices in at least the following aspects:

the invention device allows the user to insert and remove the disposable fat filtration system (Puregraft™);

the invention device allows for the closed connection to a removable disposable fat filtration system (Puregraft™);

the invention device serves as a stand that holds the disposable fat filtration system (Puregraft™) upright which allows for the manual dialysis of tissue/fluid that is inserted into the fat filtration device;

the invention device allows fluid and tissue to be inserted into the flexible filtration system and acts as a sealed chamber that prevents the flexible filtration system from compression, overexpansion and bursting; and

the invention device allows excess fluid to be removed from the flexible filtration system.

### Advantages of the Present Invention

As compared with prior art devices, the invention device offers unique advantages in at least the following aspects:

the invention device provides the user with a way to collect tissue/fluid in a closed manner that will ultimately reduce the risk of infection and contamination;

the invention device acts as a supportive exo-skeleton for a removeable filtration system for the collected tissue/fluid;

The invention device acts as a stand that supports the collection of tissue/fluid and processing of the tissue/fluid;

the invention device is able to be opened and allows the user to recover tissue/fluid that has clogged the filtration system. No other product on the market allows the user the ability to open the “exo-skeleton” and allows the user to recover the tissue; and

the invention device streamlines the surgical procedure by decreasing total operating room time and manual processes that introduce variability and exposure during the process of preparing the graft.

### DEFINITIONS

As used herein, the term tissue generally refers to tissue of an mammal such as human being. The tissue can be any kind. In some embodiments, the tissue can be a fat tissue. As used herein, the term “fat” is used interchangeably with the term “adipose”.

As used herein, the term “exo-skeleton” refers to an exterior support apparatus for the flexible filtration system. The exo-skeleton can be used to properly align, prevent over-pressuring, contain combustion due to over pressuring, and allow successful transfer of tissue into the flexible filtration system.

As used herein, the term “vacuum” generally refers to a pressure that is below the ambient pressure, which is a pressure below 1 atmosphere (1 atm). The vacuum is between a low vacuum (below but around 1 atm) and a high vacuum. Generally, the vacuum shall be one that provides sufficient vacuum effect for operating the invention device and does not injure or otherwise negatively affect the viability of the tissue. Such vacuum is generally within the range of 1 in Hg to 29.5 in Hg.

As used herein, the term “Puregraft™ fat filtration system” refers to a tissue filtration system as described in U.S. patent application publication No. 2010/0279405, the teaching of which is incorporated herein in its entirety by reference.

### Method of Fabrication

In a second aspect of the present invention, it is provided a method of forming a tissue collection device. The method comprises:

providing a vacuum chamber,
providing a disposable tissue filtration system, and
forming the tissue collection device,

wherein the vacuum chamber has a mechanism to allow the vacuum chamber to receive and open to the disposable tissue filtration system and re-seal to allow tissue/fluid to be aspirated directly in the disposable tissue filtration system via an external vacuum source.

In some embodiments of the invention method, optionally in combination with any or all the various embodiments described herein, the tissue collection device is according to any of the various embodiments disclosed herein.

The vacuum chamber can be formed of any material capable of holding a vacuum, which is defined above. In some embodiments, vacuum chamber is formed of a metallic material or a metal alloy. Preferably, the metallic material is biocompatible. Biocompatible metallic materials are known in the art. Useable metallic materials include, but are not limited to, magnesium, aluminum, silver, iron, copper, gold, platinum, titanium, zirconium, zinc, nickel, cobalt, or an alloy thereof. In some embodiments, the metallic material is stainless steel.

In some embodiments, the vacuum chamber is formed of a non-metallic material, such ceramic material or polymer. Preferably, the polymer is biocompatible, which can be biodurable or biodegradable. Examples of biodurable polymer include, but are not limited to, polyethylene, polyvinyl chloride (PVC), aliphatic polyamides, silicon, acrylonitrile butadiene styrene, acetal, aramid, polystyrene, polysulfone. Examples of biodegradable polymer include, but are not limited to, polyesters such as poly(lactic acid), polycarbonate.

The disposable tissue filtration system can be fabricated using any disposable material. In some embodiments, the disposable tissue filtration system is as described in U.S. patent application publication No. 2010/0279405, the teaching of which is incorporated herein in its entirety by reference.

### Method of Use

In a further aspect of the prevention invention, it is provided a method, which method comprising:

providing a tissue collection device, and
collection tissue via the tissue collection device,

wherein the tissue collection device is according to any of the various embodiments disclosed herein.

The invention device can be used to collect any tissue. Preferably, the tissue is a fat adipose. In some embodiments, the vacuum chamber can be designed specifically for multiple sizes of Puregraft™ fat filtration systems. The device(s) will be biologically and mechanically tested prior to regulatory clearance and eventually commercially sold.

The system can be used to collect multiple types of tissue/fluid not just adipose and fluids removed via the liposuction process in a closed manner.

### Examples

The following examples illustrate the present invention and shall not be construed to limit the scope of the present invention.

Examples of the invention device were construed according to the description above.

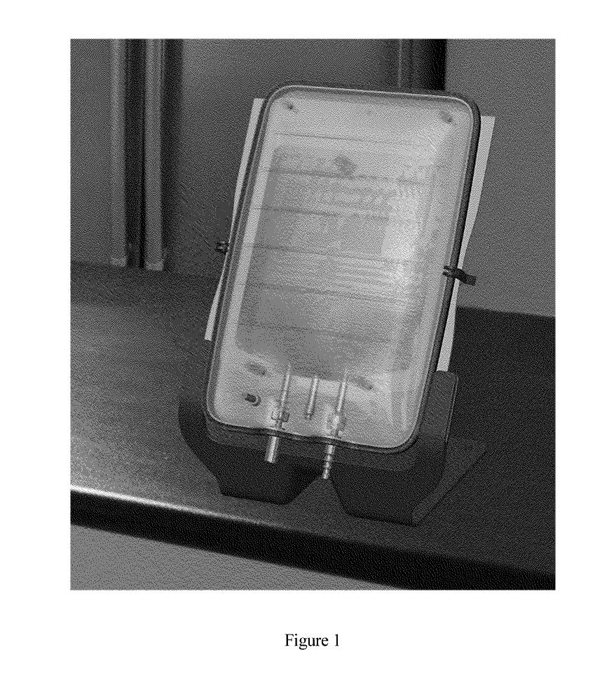

FIG. 1 shows a photo image of one of such examples.

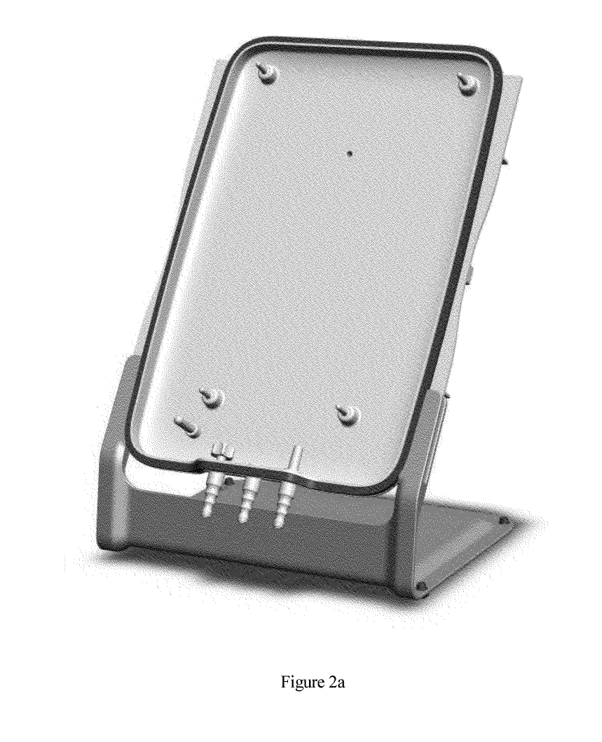

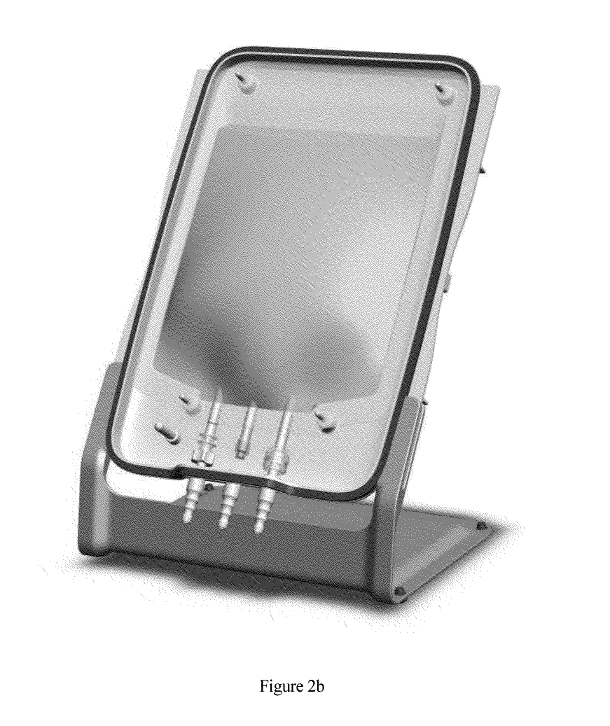

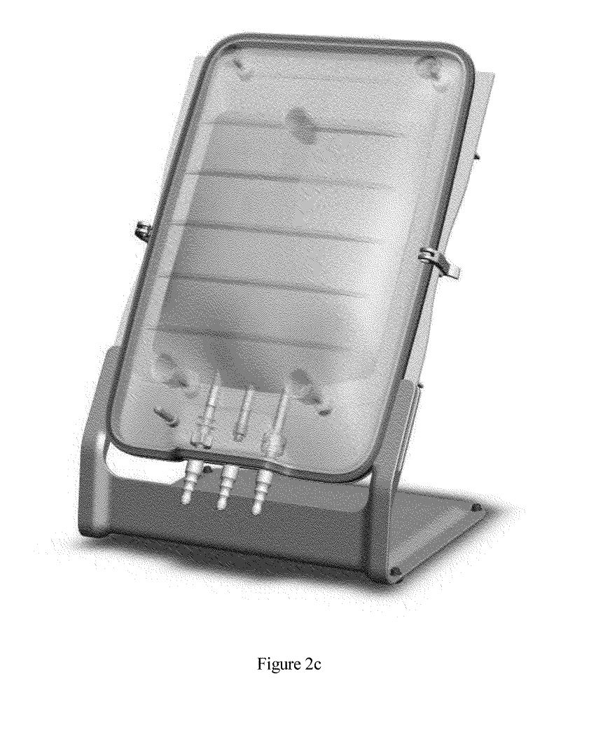

FIGS. 2a-2c show photo images of an exemplary device prior to —, during —, and after its use in a tissue collection operation.

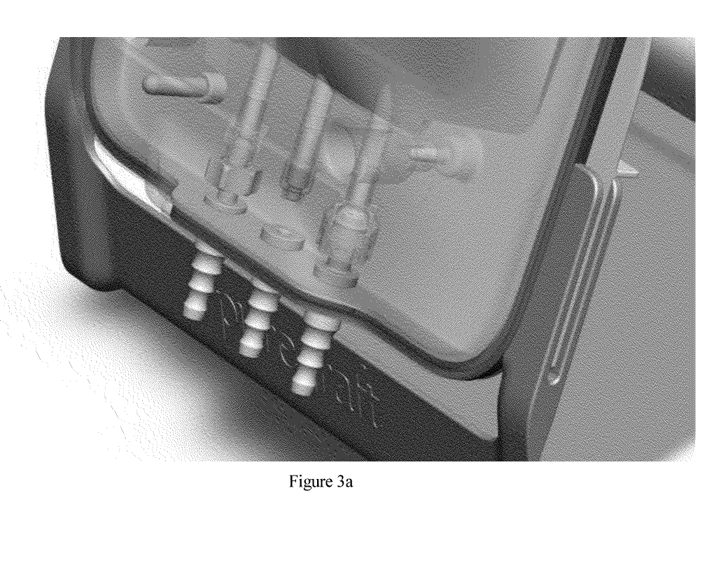

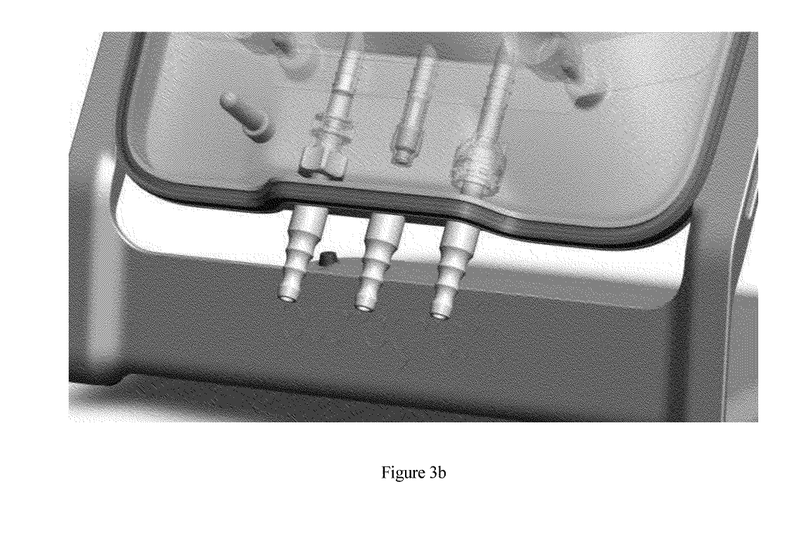

FIGS. 3a and 3b show photo images of the vacuum connection mechanisms of an exemplary device of invention.

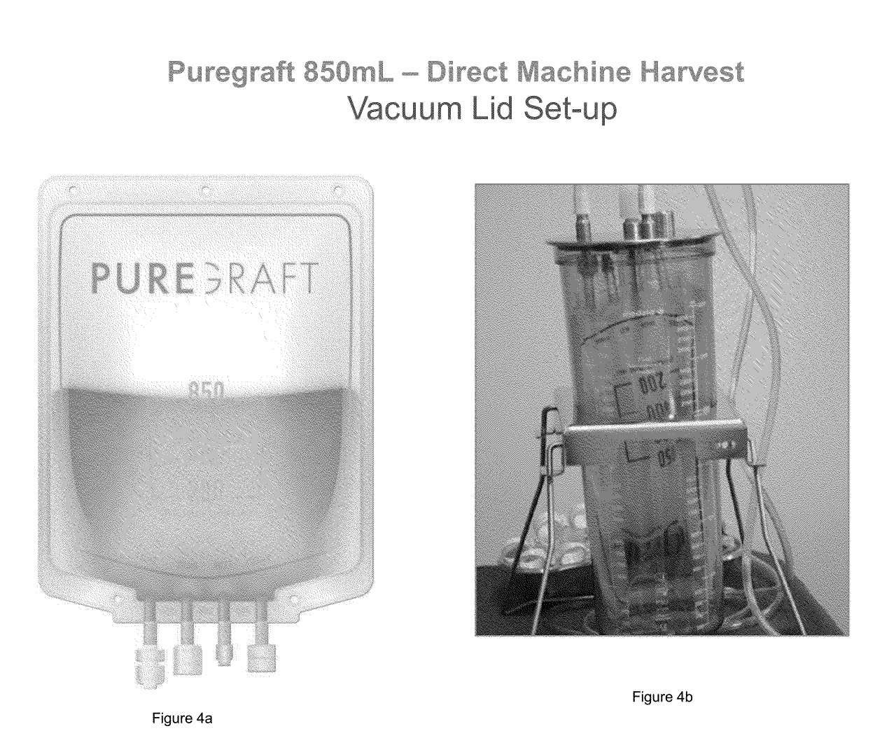

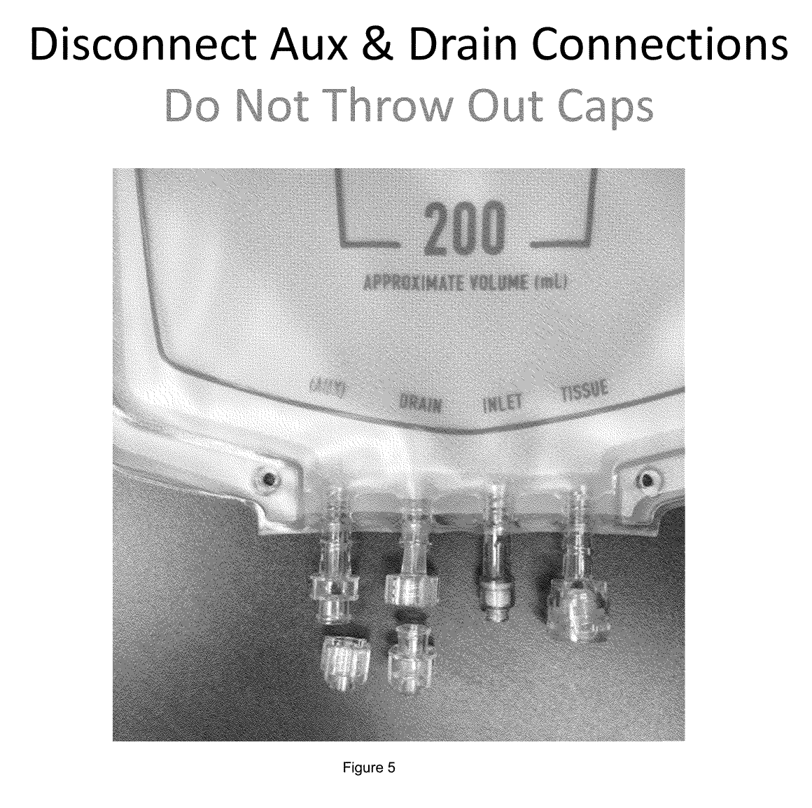

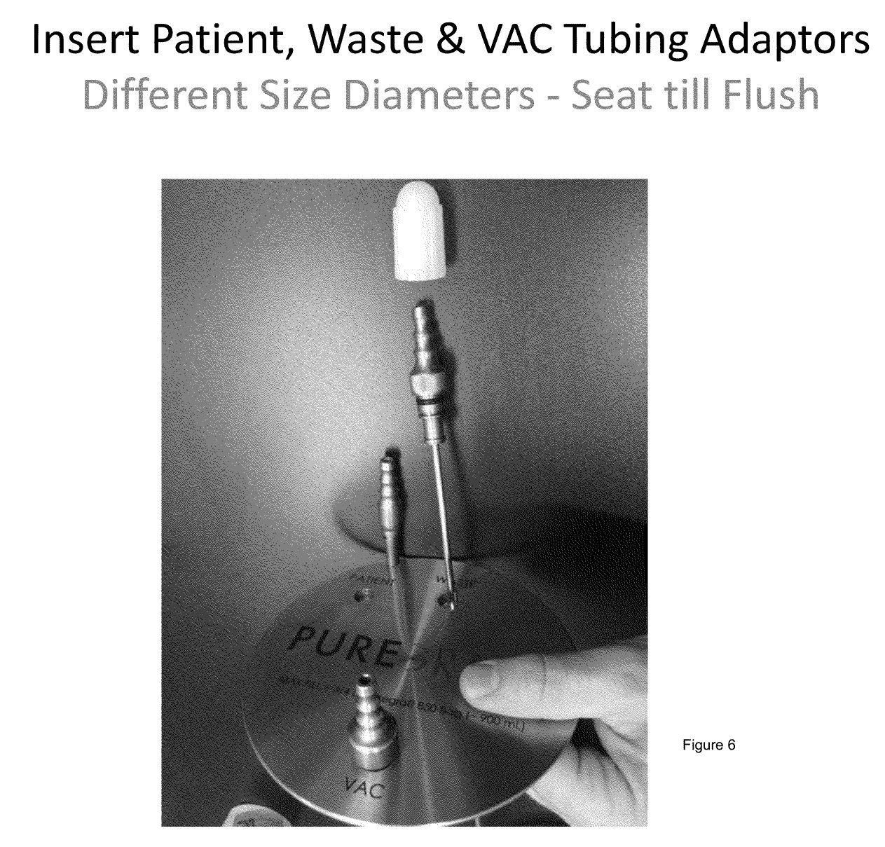

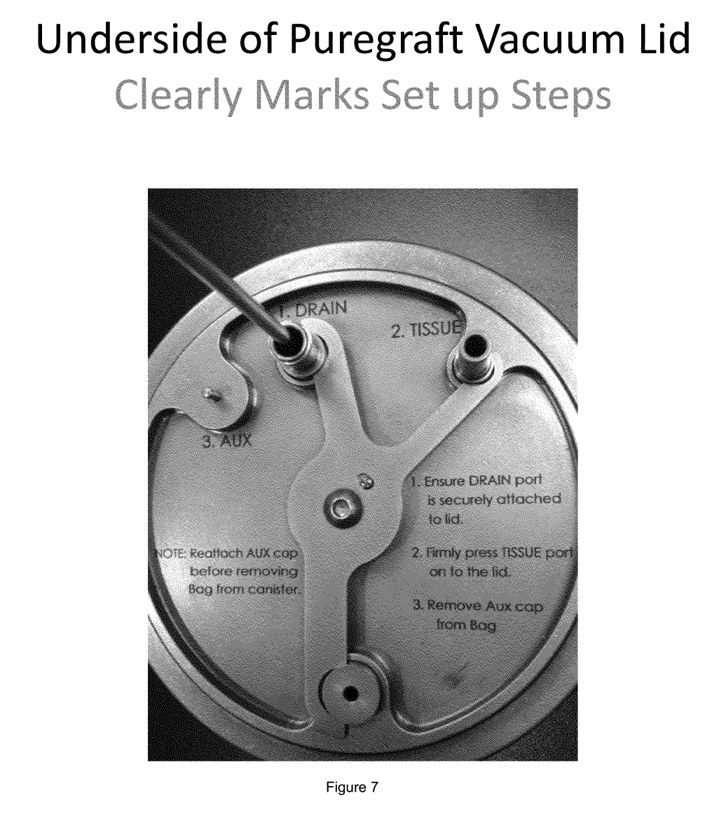

FIGS. 4-16 show photo images of using an exemplary device (Puregraft 850 ml) in a tissue collection operation under vacuum-direct machine harvest operation. FIG. 4a shows a Puregraft 850 ml bag filled with adipose tissue. FIG. 4b shows a Puregraft 850 ml in full set up on a canister with a vacuum lid, the canister bearing volume marks. FIG. 5 shows the connection ports of a Puregraft 850 ml bag, which include an auxiliary (Aux) port, drain port, an inlet port, and a tissue port. FIG. 6 shows the top side of a vacuum lid, which includes port to allow insertion of adaptors to vacuum tubing, patient tubing, and waste tubing. FIG. 7 shows the underside of a vacuum lid, which shows Aux port, drain port, an and a tissue port, and operation notes. The operation notes read:

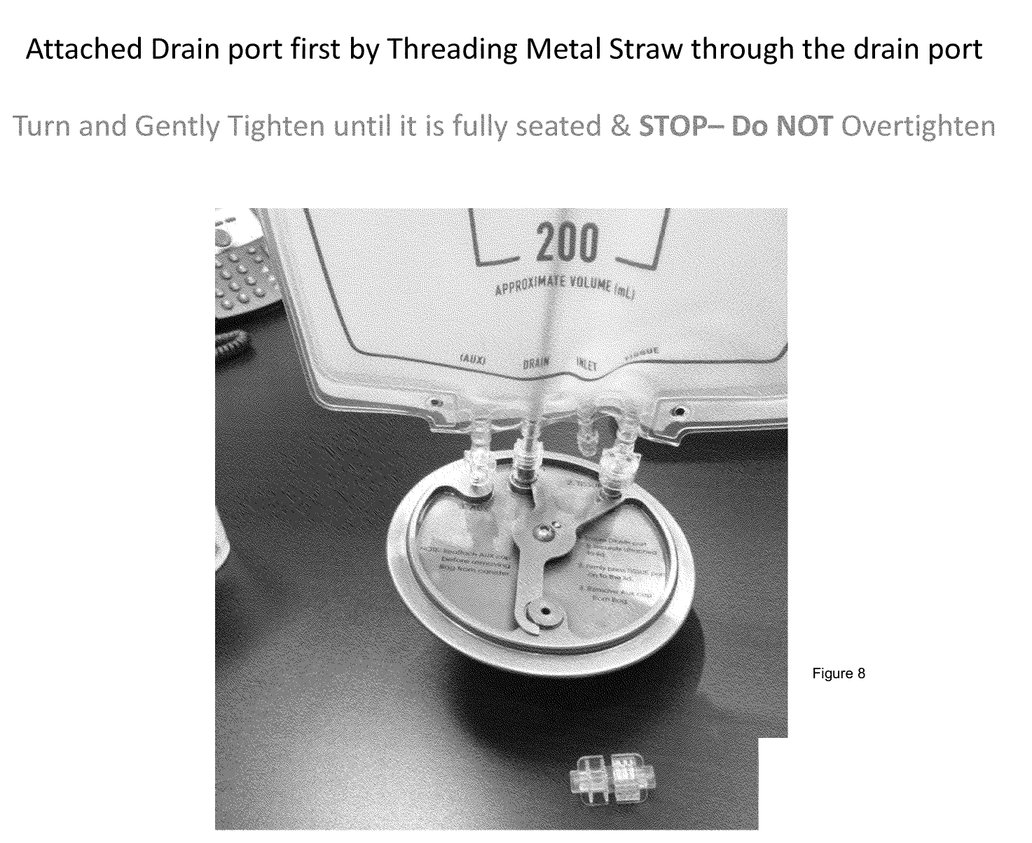

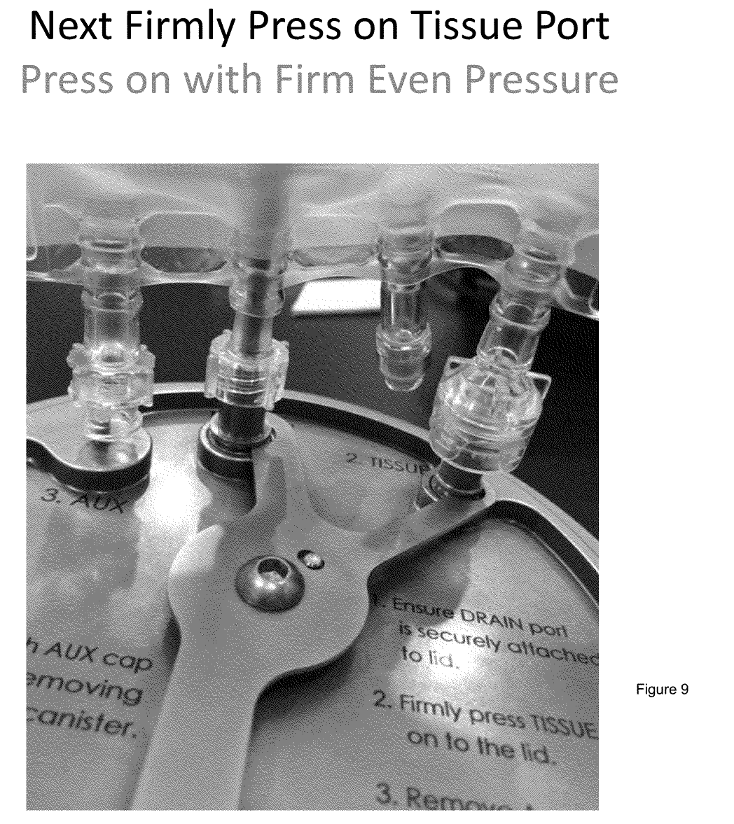

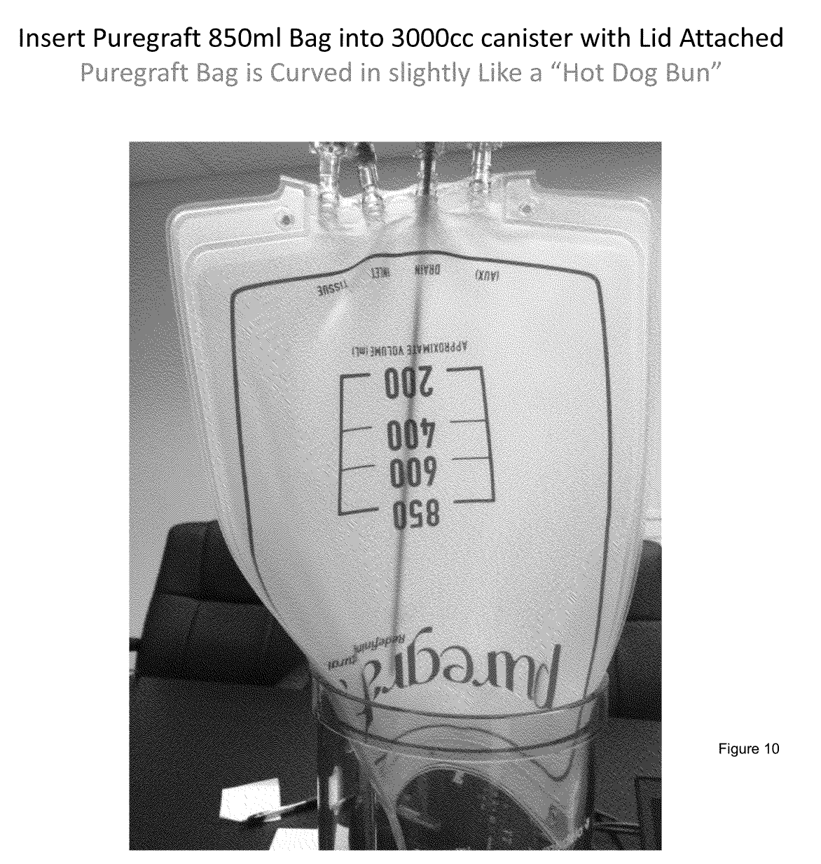

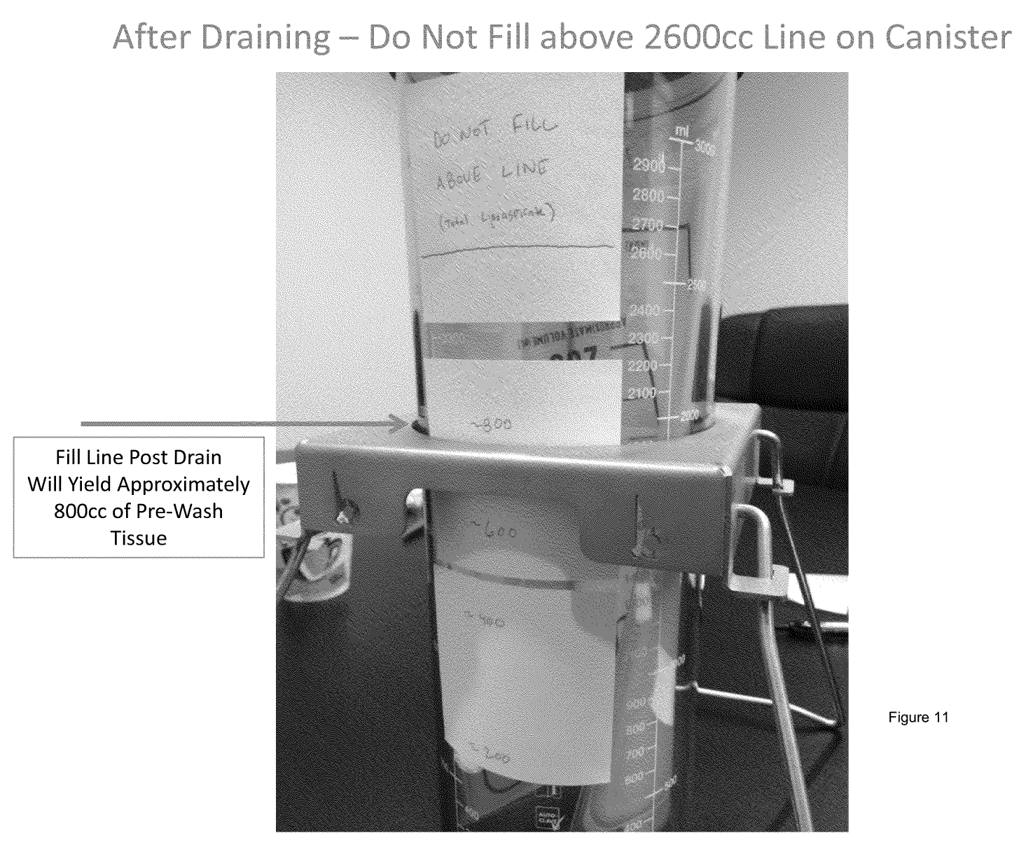

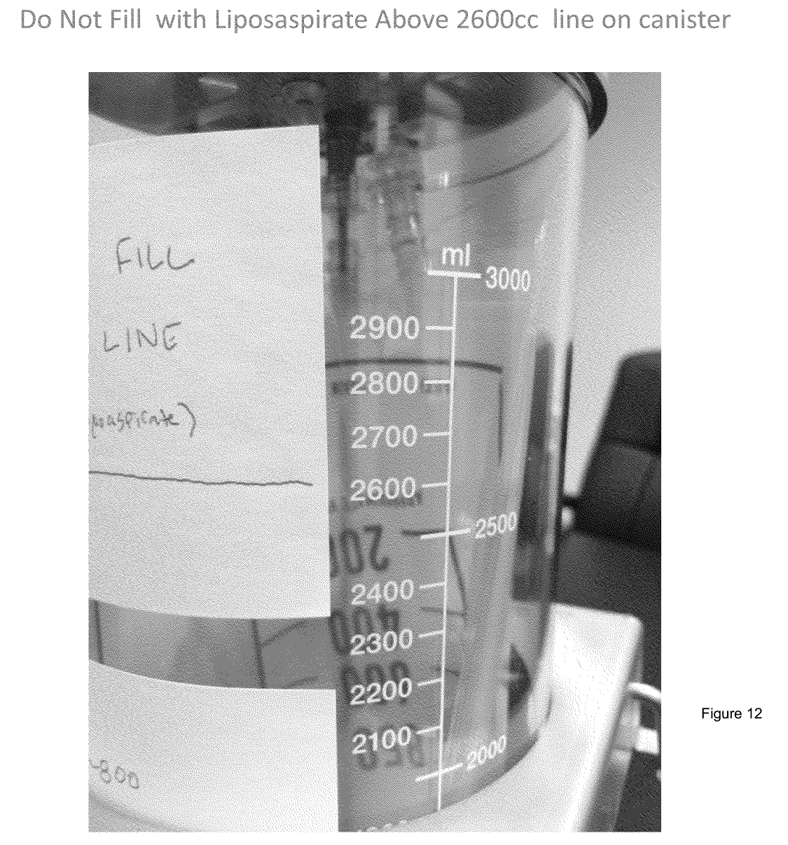

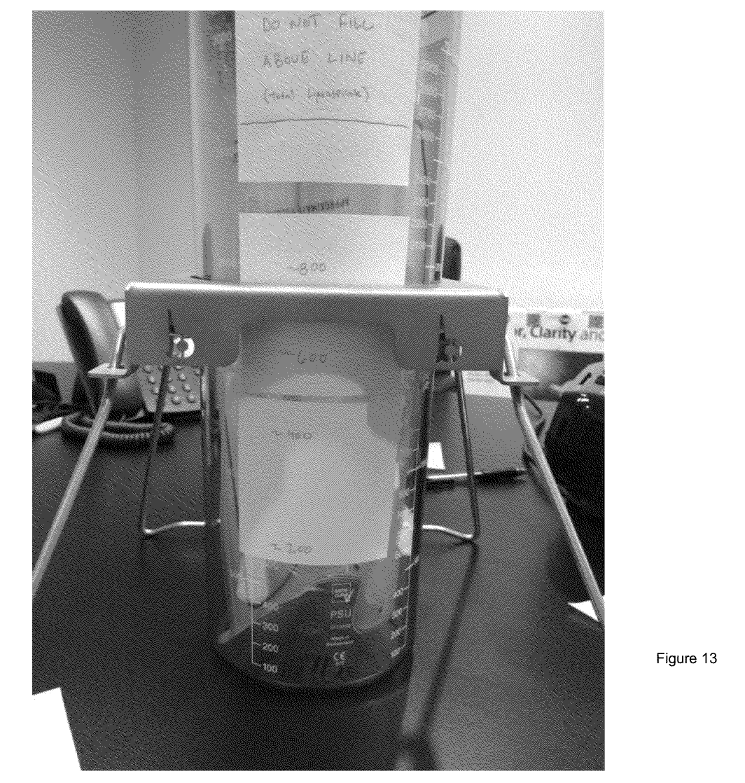

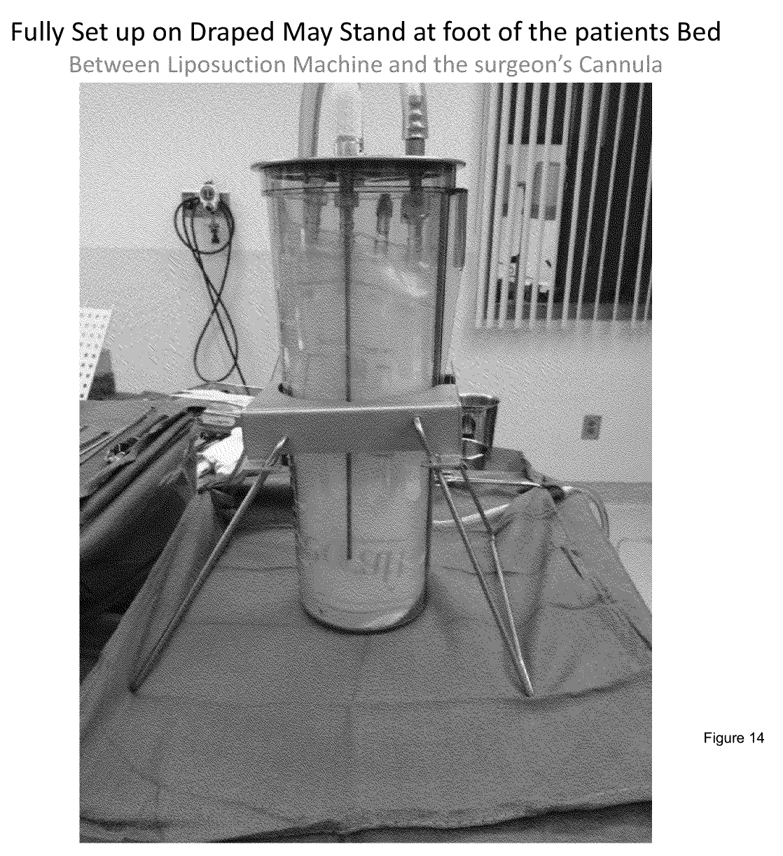

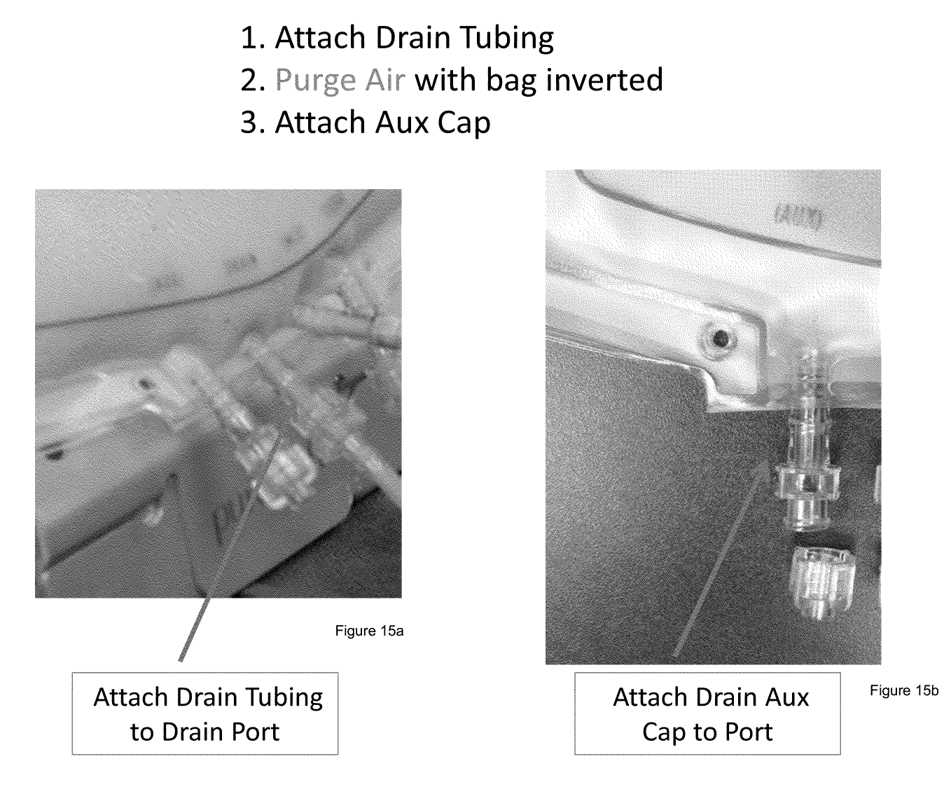

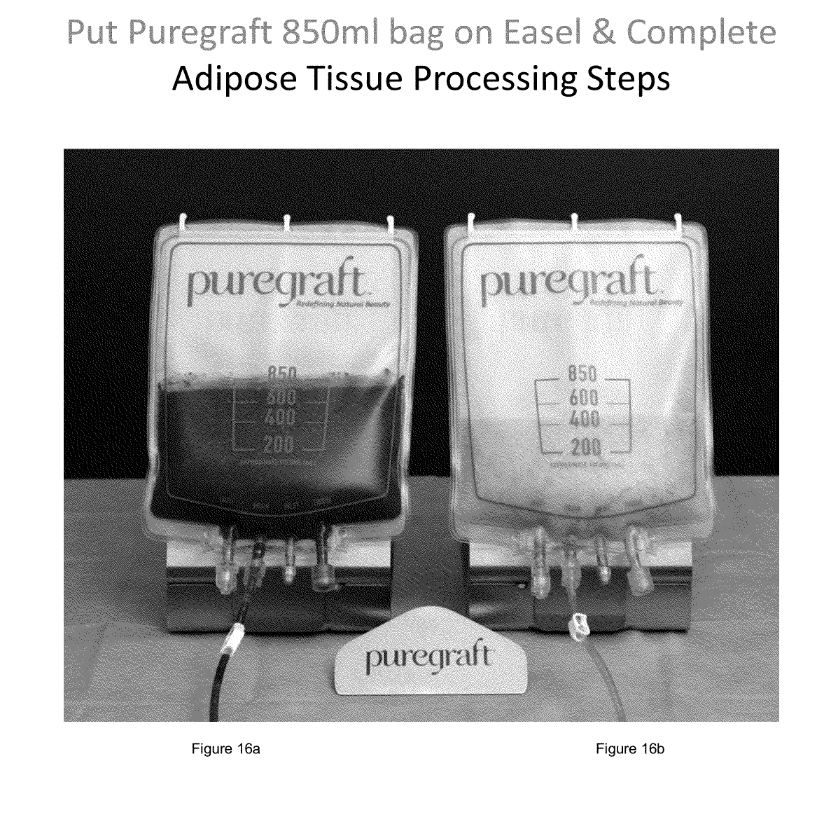

1. Ensure Drain port is securely attached to lid,
2. Firmly press Tissue port on to the lid,
3. Remove Aux cap from bag; and
Note: Reattach Aux cap before removing bag from canister.
FIG. 8 shows instructions to attach the drain port of a Puregraft 850 ml bag to a vacuum lid. Such instructions read:
—Attaching Drain port first by threading metal straw through the drain port—, and
—Turn and gently tighten until its fully seated & STOP—Do not Overtighten —.
FIG. 9 shows the step to attach the tissue port of a Puregraft 850 ml bag to a vacuum lid, which states—Next Firmly Press on Tissue Port —, and—Press On with Firm Even Pressure—.
FIG. 10 shows that a Puregraft 850 ml bag is inserted into a 3000 cc canister with Lid attached.
FIG. 11 shows a Puregraft 850 ml bag in a canister. FIG. 11 also shows the proper filling level after draining and states—After draining—Do not Fill above 2600 cc Line on Canister —, and—Fill Line post Drain Will Yield Approximately 800 cc of Pre-Wash Tissue—.
FIG. 12 again shows a Puregraft 850 ml bag in a canister. Like FIG. 11, FIG. 12 also shows the proper filling level with the statement—Do Not Fill with Liposaspirate Above 2600 cc Line on Canister —.
FIG. 13 shows a Puregraft 850 ml bag in a canister on a drapped May Stand.
FIG. 14 shows a Puregraft 850 ml bag in a canister on a drapped May Stand at the foot of a patient's bed between the Liposuction machine and the surgeon's cannula.
FIG. 15a shows a Puregraft 850 ml bag filled with adipose tissue indication to attach drain port to drain port. FIG. 15b shows an indication to attach drain aux cap to port.
FIG. 16a shows a Puregraft 850 ml bag filled with approximately 800 ml pre-washed adipose tissue. FIG. 16b shows a Puregraft 850 ml bag filled with approximately 500 ml washed adipose tissue.

Those skilled in the art will know, or be able to ascertain, using no more than routine experimentation, many equivalents to the specific embodiments of the invention described herein. These and all other equivalents are intended to be encompassed by the following claims.

1. A tissue collection device, comprising a vacuum chamber or vacuum lid in conjunction with a disposable tissue filtration system, wherein the vacuum chamber or vacuum lid has a mechanism to allow the vacuum chamber to receive and open to the disposable tissue filtration system and re-seal to allow tissue/fluid to be aspirated directly in the disposable tissue filtration system via an external vacuum source.

2. The tissue collection device of claim 1, wherein the vacuum chamber is a disposable vacuum chamber.

3. The tissue collection device of claim 2, wherein the disposable tissue filtration system is physically bonded into the disposable vacuum chamber such that the tissue collection device as a whole is a single use, disposable device.

4. The tissue collection device of claim 1, wherein the vacuum chamber or vacuum lid is formed of a metal material.

5. The tissue collection device of claim 1, wherein the vacuum chamber or vacuum lid is formed of stainless steel.

6. The tissue collection device of claim 2, wherein the vacuum chamber is formed of a sterilizable and disposable material.

7. The tissue collection device of claim 6, wherein the disposable material is a polymer.

8. The tissue collection device of claim 7, wherein the polymer is poly(vinyl chloride) (PVC) or polycarbonate.

9. The tissue collection device of claim 3, wherein the vacuum chamber is formed of a sterilizable and disposable material.

10. The tissue collection device of claim 9, wherein the disposable material is a polymer.

11. The tissue collection device of claim 10, wherein the polymer is poly(vinyl chloride) (PVC) or polycarbonate.

12. The tissue collection device of claim 1, wherein the disposable tissue filtration system comprises a disposable fat tissue filtration system.

13. The tissue collection device of claim 2, wherein the disposable tissue filtration system is a disposable fat tissue filtration system.

14. The tissue collection device of claim 3, wherein the disposable tissue filtration system is a disposable fat tissue filtration system.

15. The tissue collection device of claim 1, wherein the disposable tissue filtration system comprises a disposable bag in connection with the vacuum lid.

16. A method of forming a tissue collection device, comprising:
providing a vacuum chamber or a vacuum lid,
providing a disposable tissue filtration system, and
forming the tissue collection device,
wherein the vacuum chamber or vacuum lid has a mechanism to allow the vacuum chamber to receive and open to the disposable tissue filtration system and re-seal to allow tissue/fluid to be aspirated directly in the disposable tissue filtration system via an external vacuum source.

17. A method, comprising:
providing a tissue collection device, and
collection tissue via the tissue collection device,
wherein the tissue collection device comprises a vacuum chamber or vacuum lid in conjunction with a disposable tissue filtration system, wherein the vacuum chamber has a mechanism to allow the vacuum chamber to receive and open to the disposable tissue filtration system and re-seal to allow tissue/fluid to be aspirated directly in the disposable tissue filtration system via an external vacuum source.
